(19) United States
(12) Patent Application Publication
(10) Pub. No.: US 2025/0161314 A1
(43) Pub. Date: May 22, 2025

(54) METHOD FOR TREATMENT OR IMPROVEMENT OF NON-ALCOHOLIC FATTY LIVER DISEASE AND OBESITY BY LUMICHROME

(71) Applicant: CATHAY GENERAL HOSPITAL, Taipei City (TW)

(72) Inventor: TING-LIN YEN, New Taipei City (TW)

(21) Appl. No.: 18/413,542

(22) Filed: Jan. 16, 2024

(30) Foreign Application Priority Data
Nov. 21, 2023 (TW) 112144931

(51) Int. Cl. A61K 31/525; A61P 1/16; A61P 3/04

(52) U.S. Cl. CPC A61K 31/525; A61P 1/16; A61P 3/04

(57) ABSTRACT

The present invention provides a method for a treatment or improvement of non-alcoholic fatty liver disease (NAFLD), comprising administering a composite to a subject in need thereof, wherein the composite comprises lumichrome. Lumichrome can improve fatty liver and weight gains resulting from a high-fat diet, and improve the liver function index of aspartate transaminase, alanine transaminase or lactate dehydrogenase. Further, lumichrome can also inhibit the increase of visceral fat and lipid droplets accumulation in the liver, and inhibit the mRNA expression increase of Acta2, CD36 or FABP1. Therefore, lumichrome is effective for the treatment or improvement of non-alcoholic fatty liver disease and/or obesity.

### STATEMENT REGARDING SEQUENCE LISTING

The sequence listing associated with this application is provided in text format in lieu of a paper copy and is hereby incorporated by reference into the specification. The name of the XML file containing the sequence listing is sl.xml. The XML file is 2,797,488 bytes; was created on Jan. 16, 2024; and is being submitted electronically via Patent Center.

### CROSS-REFERENCE TO RELATED APPLICATION

Pursuant to 35 U.S.C. § 119(a), this application claims the benefits of the priority to Taiwan Patent Application No. 112144931, filed on Nov. 21, 2023, which is incorporated by reference herein in its entirety.

### BACKGROUND OF THE INVENTION

### 1. Field of the Invention

The present invention relates to a method for a treatment or improvement of non-alcoholic fatty liver disease or obesity, especially by administering lumichrome to a subject in need thereof.

### 2. Description of the Prior Arts

Non-alcoholic fatty liver disease (NAFLD) is a common disease. Normally, the liver contains approximately 3% to 5% fat. According to the guidelines published by the American Association for the Study of Liver Diseases, NAFLD is defined as the presence of ≥5% hepatic steatosis either by liver imaging or histology, and lacks secondary causes of hepatic fat accumulation such as alcohols, drugs or viruses. NAFLD is characterized by an imbalance in the synthesis and utilization of fat in the liver, and results in excessive accumulation of triglycerides (TG). Without effective control, NAFLD will eventually develop into non-alcoholic steatohepatitis (NASH), cirrhosis and end-stage liver disease.

Currently, the generally accepted treatments for NAFLD are weight loss and fat intake reduction. Possible drugs available comprise hypoglycemic drugs, hypolipidemic drugs (such as statin drugs), anti-inflammatory drugs, antioxidants (such as vitamin E), etc. However, all these drugs lack large-scale clinical studies or have potential side effects, so currently no drugs are clinically approved for the treatment of NAFLD. Therefore, there is an urgent need to further study and develop the treatment for NAFLD.

### SUMMARY OF THE INVENTION

In view of the aforementioned problem in the current technology, the present invention provides a method for a treatment or improvement of non-alcoholic fatty liver disease (NAFLD), comprising administering a composite to a subject in need thereof, wherein the composite comprises lumichrome, which can effectively fight against NAFLD.

To achieve the aforementioned objective, the present invention provides a method for a treatment or improvement of non-alcoholic fatty liver disease (NAFLD), comprising administering a composite to a subject in need thereof, wherein the composite comprises lumichrome.

The example of the present invention proves that lumichrome can (1) improve fatty liver resulting from a high-fat diet; (2) inhibit weight gains resulting from a high-fat diet; (3) inhibit the increase of aspartate transaminase (AST), alanine transaminase (ALT) and lactate dehydrogenase (LDH) resulting from a high-fat diet; (4) inhibit the increase of visceral fat or body fat resulting from a high-fat diet; (5) inhibit lipid droplets (LDs) accumulation in the liver resulting from a high-fat diet; (6) inhibit the mRNA expression increase of Acta2, which relates to liver fibrosis, resulting from a high-fat diet; inhibit the mRNA expression increase of CD36 or FABP1, which both relate to fat uptake by liver cells, resulting from a high-fat diet. Therefore, lumichrome is effective for the treatment or improvement of non-alcoholic fatty liver disease.

To achieve the aforementioned objective, the present invention provides a method for the treatment or improvement of obesity, comprising administering a composite to a subject in need thereof, wherein the composite comprises lumichrome.

According to the present invention, lumichrome is a photosensitizer, and has a chemical formula of C₁₂H₁₀N₄O₂ and a CAS number of 1086-80-2.

Preferably, the composite is a medical composite or a food composite.

Preferably, the medical composite comprises an effective dose of lumichrome and a pharmaceutically acceptable carrier.

In the present invention, the “effective dose” refers to the amount that is effective to achieve the desirable treatment or improvement of NAFLD or obesity within a required period of time. According to the present invention, by administering a specific amount range of said “effective dose” of lumichrome, lumichrome can (1) improve fatty liver resulting from a high-fat diet; (2) inhibit weight gains resulting from a high-fat diet; (3) inhibit the increase of AST, ALT and LDH resulting from a high-fat diet; (4) inhibit the increase of visceral fat or body fat resulting from a high-fat diet; (5) inhibit lipid droplets accumulation in the liver resulting from a high-fat diet; and (6) inhibit the mRNA expression increase of Acta2, CD36 or FABP1 resulting from a high-fat diet.

Preferably, the subject to be administered with the medical composite is a mammal, such as a human. An effective dose of lumichrome is 0.01 mg/kg/day to 2 mg/kg/day. More preferably, the effective dose of lumichrome is 0.05 mg/kg/day to 1 mg/kg/day. For example, the effective dose of lumichrome can be 0.2 mg/kg/day to 0.5 mg/kg/day.

More preferably, the effective dose of lumichrome for a human is 0.07 mg/kg/day to 0.3 mg/kg/day. This dose range is calculated based on the experimental doses for the mice in the example of the present invention and according to “Estimating the maximum safe starting dose in initial clinical trials for therapeutics in adult healthy volunteers” published by the US Food and Drug Administration in 2005.

In the present invention, “a pharmaceutically acceptable carrier” comprises, but is not limited to, a solvent, an emulsifier, a suspending agent, a decomposer, a binding agent, an excipient, a stabilizing agent, a diluent, a gelling agent, a lubricant, a surfactant, other similar carriers, or a carrier that is suitable for the present invention.

In the present invention, “a medical composite” can be in various forms. Such forms comprise, but are not limited to, a liquid, a semi-solid or a solid dosage form. For example, the medical composite can be a solution, an emulsion, a suspension, a powder, a tablet, a pill, a lozenge, a troche, a chewing gum, a capsule, a liposome, a suppository, other similar dosage forms, or a dosage form that is suitable for the present invention.

Preferably, the medical composite is an enteral or a parenteral dosage form.

More preferably, the enteral dosage form is an oral dosage form. The oral dosage form is a solution, an emulsion, a suspension, a powder, a tablet, a pill, a lozenge, a troche, a chewing gum or a capsule.

Preferably, the food composite has an efficacy to inhibit body fat formation.

Preferably, the food composite is a nutraceutical, a dietary supplement or a functional supplement.

The present invention has the advantages as follows: without loss of muscle mass or muscular endurance, lumichrome can decrease weight gains, decrease increased body fat, decrease increased visceral fat, improve fatty liver, and inhibit lipid droplets accumulation in the liver resulting from a high-fat diet. Therefore, lumichrome has excellent therapeutic effects on non-alcoholic fatty liver disease and/or obesity.

### BRIEF DESCRIPTION OF THE DRAWINGS

FIG. 1 shows the influences on the body size and liver appearance of mice after the treatment with a high-fat diet and lumichrome.

FIG. 2 shows the influences on the weight gains of mice after the treatment with a high-fat diet and lumichrome, wherein * indicates p<0.05 compared with the High-fat diet group, ** indicates p<0.01 compared with the High-fat diet group, and *** indicates p<0.001 compared with the High-fat diet group.

FIG. 3 shows the influences on the grip strengths of mice after the treatment with a high-fat diet and lumichrome.

FIG. 4A shows the influences on AST in the serum of mice after the treatment with a high-fat diet and lumichrome, wherein ### indicates p<0.001 compared with the Control group, and * indicates p<0.05 compared with the High-fat diet group.

FIG. 4B shows the influences on ALT in the serum of mice after the treatment with a high-fat diet and lumichrome, wherein ### indicates p<0.001 compared with the Control group, and *** indicates p<0.001 compared with the High-fat diet group.

FIG. 4C shows the influences on LDH in the serum of mice after the treatment with a high-fat diet and lumichrome, wherein ## indicates p<0.01 compared with the Control group, and * indicates p<0.05 compared with the High-fat diet group.

FIG. 5 shows the influences on the epididymal fat weights of mice after the treatment with a high-fat diet and lumichrome, wherein ### indicates p<0.001 compared with the Control group, and *** indicates p<0.001 compared with the High-fat diet group.

FIG. 6 shows the influences on the liver weights of mice after the treatment with a high-fat diet and lumichrome, wherein ## indicates p<0.01 compared with the Control group, and * indicates p<0.05 compared with the High-fat diet group.

FIG. 7 shows the influences on the lipid amount in the liver of mice after the treatment with a high-fat diet and lumichrome, wherein ### indicates p<0.001 compared with the Control group, and *** indicates p<0.001 compared with the High-fat diet group.

FIG. 8A shows the photos at low magnification of the liver sections of mice after hematoxylin and eosin staining (H/E staining), wherein the scale bar indicates 210 microns, and the mice were treated with a high-fat diet and lumichrome.

FIG. 8B shows the photos at high magnification of the liver sections of mice after H/E staining, wherein the scale bar indicates 110 microns, and the mice were treated with a high-fat diet and lumichrome.

FIG. 9A shows the photos at low magnification of the liver sections of mice after Oil red O (ORO) staining, wherein the scale bar indicates 210 microns, and the mice were treated with a high-fat diet and lumichrome.

FIG. 9B shows the photos at high magnification of the liver sections of mice after ORO staining, wherein the scale bar indicates 110 microns, and the mice were treated with a high-fat diet and lumichrome.

FIG. 10 shows the influences on the relative mRNA expression level of Acta2 in the liver of mice after the treatment with a high-fat diet and lumichrome, wherein the Control group serves as a standard, # indicates p<0.05 compared with the Control group, and *** indicates p<0.001 compared with the High-fat diet group.

FIG. 11 shows the influences on the relative mRNA expression level of CD36 in the liver of mice after the treatment of a high-fat diet and lumichrome, wherein the Control group serves as a standard, ### indicates p<0.001 compared with the Control group, and ** indicates p<0.01 compared with the High-fat diet group.

FIG. 12 shows the influences on the relative mRNA expression level of FABP1 in the liver of mice after the treatment with a high-fat diet and lumichrome, wherein the Control group serves as a standard, # indicates p<0.05 compared with the Control group, ### indicates p<0.001 compared with the Control group, and * indicates p<0.05 compared with the High-fat diet group.

### DETAILED DESCRIPTION OF THE PREFERRED EMBODIMENTS

The present invention is further explained through the following embodiments and the accompanied drawings. A person having ordinary skill in the art can easily understand the advantages and efficacies achieved by the present invention.

### Example: Animal Experiment with a Treatment of a High-Fat Diet and Lumichrome

Eight-week-old C57BL/6 male mice (purchased from the National Laboratory Animal Center, Taiwan) were housed in a breeding room with a 12-hour light-dark cycle, and were randomly divided into the following groups for experiments, wherein each group had 6 mice.
(i) Control group: a control diet, which was a rodent diet with 10 kcal % fat (D12450Hi, Research Diets, Inc., NJ, USA), was supplied ad libitum to mice for 32 weeks, and the 10 kcal % fat indicated that fat accounted for 10% calories (kcal) in the total calories of the rodent diet.
(ii) Control-lumichrome in low dose group: the control diet was supplied ad libitum to mice for 32 weeks, and extra oral gavage of 200 μL solution of lumichrome dissolved in PBS twice a day was initiated at 21th week during the 32 weeks, wherein lumichrome in a dose of 0.4 mg/kg was provided each time, and the daily dose of lumichrome was 0.8 mg/kg/day.
(iii) Control-lumichrome in high dose group: the control diet was supplied ad libitum to mice for 32 weeks, and extra oral gavage of 200 μL solution of lumichrome dissolved in PBS twice a day was initiated at 21th week during the 32 weeks, wherein lumichrome in a dose of 2 mg/kg was provided each time, and the daily dose of lumichrome was 4 mg/kg/day.
(iv) High-fat diet group: a high-fat diet (HFD), which was a rodent diet with 45 kcal % fat (D12451i, Research Diets, Inc., NJ, USA), was supplied ad libitum to mice for 32 weeks.
(v) HFD-lumichrome in low dose group: the HFD was supplied ad libitum to mice for 32 weeks, and extra oral gavage of 200 μL solution of lumichrome dissolved in PBS twice a day was initiated at 21th week during the 32 weeks, wherein lumichrome in a dose of 0.4 mg/kg was provided each time, and the daily dose of lumichrome was 0.8 mg/kg/day.
(vi) HFD-lumichrome in high dose group: the HFD was supplied ad libitum to mice for 32 weeks, and extra oral gavage of 200 μL solution of lumichrome dissolved in PBS twice a day was initiated at 21th week during the 32 weeks, wherein lumichrome in a dose of 2 mg/kg was provided each time, and the daily dose of lumichrome was 4 mg/kg/day.

All groups were further subjected to the following tests:
(1) All groups were subjected to a weight measurement every week, and sacrificed after 32 weeks by excessive anesthesia through isoflurane.
(2) All groups were subjected to a grip strength test to measure the grip strengths of mice by a grip strength meter (REZ-5, Digitech Co., Ltd, Osaka. JP) before anesthesia.
(3) All groups were firstly subjected to a general anesthesia with 3% isoflurane before the mice in all groups were eventually sacrificed, and the bloods thereof were collected from the inferior vena cava of the mice to separate the serum. The serum was further analyzed by a dry chemistry auto analyzer (DRI-CHEM NX500i, Fujifilm, Tokyo, Japan) to measure the following blood biochemical values: AST, ALT and LDH.
(4) The mice in all groups were eventually sacrificed by excessive anesthesia, and both the epididymal fat and liver thereof were collected for a weight measurement, wherein the liver was divided into three parts, and a first part thereof, the first liver tissue, was further tested to determine the lipid amount in the liver by a lipid extraction kit (Chloroform-Free) (ab211044, Abeam, Cambridge, UK).
(5) A second part of the liver, the second liver tissue, was soaked in 4% formaldehyde (10% formalin) for frozen section, and the obtained liver tissue section was further subjected to (1) H/E staining and (2) Oil red O (ORO) staining for lipids (Bio-check laboratories, Taipei, Taiwan).
(6) A third part of the liver, the third liver tissue, was subjected to mRNA extraction, and the extracted mRNA was further subjected to quantitative polymerase chain reaction (qPCR). Further, (1) by qPCR with the primers of SEQ ID NO:1 and SEQ ID NO:2, the relative mRNA expression level of Acta2, which relates to liver fibrosis, in the liver in all groups was determined. (2) by qPCR with the primers of SEQ ID NO:3 and SEQ ID NO:4, the relative mRNA expression level of CD36, which affects fat uptake by liver cells, in all groups was determined; and (3) by qPCR with the primers of SEQ ID NO:5 and SEQ ID NO:6, the relative mRNA expression level of FABP1, which affects fat uptake by liver cells, in all groups was determined.

The present invention used a statistic and graphing software (prism ver. 6) for data analysis. The data of the numerical mean±standard deviation (SD) of the independent samples in all six groups were firstly obtained, and further subjected to One-way ANOVA for statistics. The post-hoc test was Tukey multiple comparison test. The differences between the groups were analyzed separately, and a p value less than 0.05 indicated a statistically significant difference.

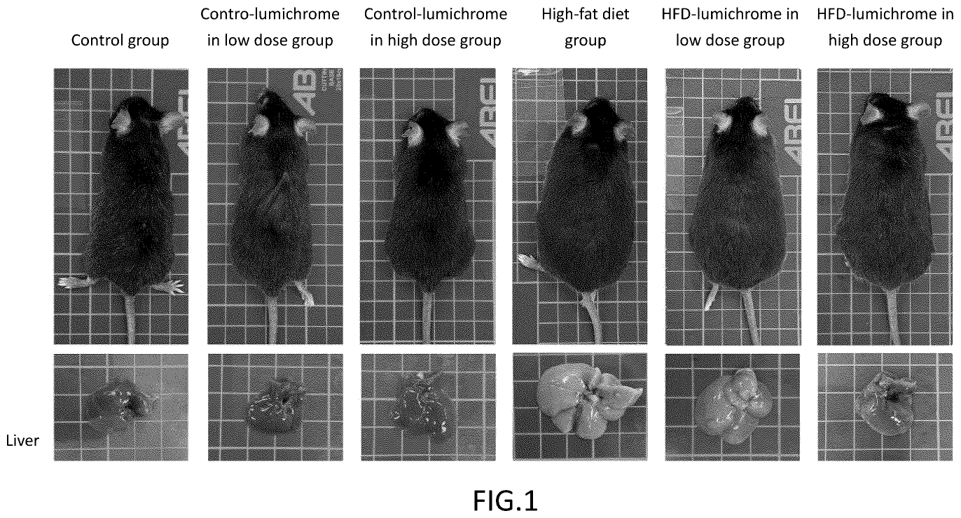

As shown in FIG. 1, in comparison between all the three control groups (Control group, Control-lumichrome in low dose group and Control-lumichrome in high dose group) and all the three high-fat diet groups (High-fat diet group, HFD-lumichrome in low dose group and HFD-lumichrome in high dose group), one can find that the high-fat diet increased the body size of mice, and caused the liver tissues to turn white and show the appearance of a fatty liver.

Among all the three high-fat diet groups, one may find that lumichrome in both low and high doses can improve the appearance of a fatty liver and regain a red color, and inhibit the increase of the body size of mice. In particular, the HFD-lumichrome in high dose group demonstrated a more obvious efficacy to ameliorate the body size increase of mice resulting from the high-fat diet, and to improve the appearance of a fatty liver to regain a blood red color.

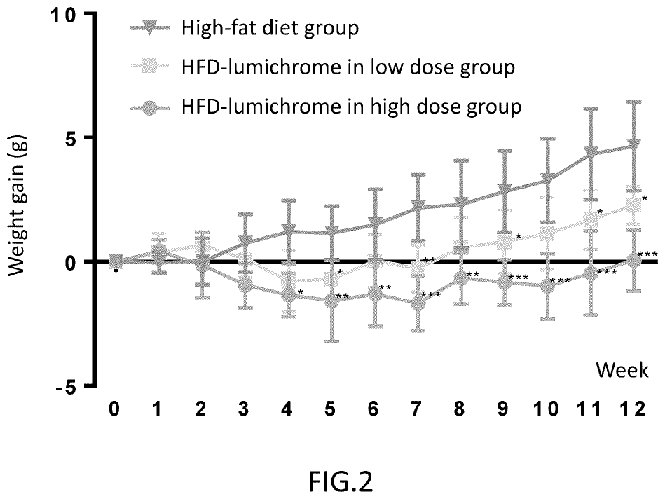

As shown in FIG. 2, first, week 0 is the time to provide lumichrome to both the HFD-lumichrome in low dose group and the HFD-lumichrome in high dose group. Second, the High-fat diet group demonstrated a positive value of weight gain for 12 weeks, so one can find that the high-fat diet increased the weight of mice. Third, lumichrome in both low and high doses can ameliorate weight gain increase of mice resulting from the high-fat diet.

In particular, the weight gains of mice in the HFD-lumichrome in high dose group no longer increased greatly at and after the fourth week after providing lumichrome, and there were statistically significant differences of weight gains between the High-fat diet group and the HFD-lumichrome in high dose group at and after the fourth week after providing lumichrome.

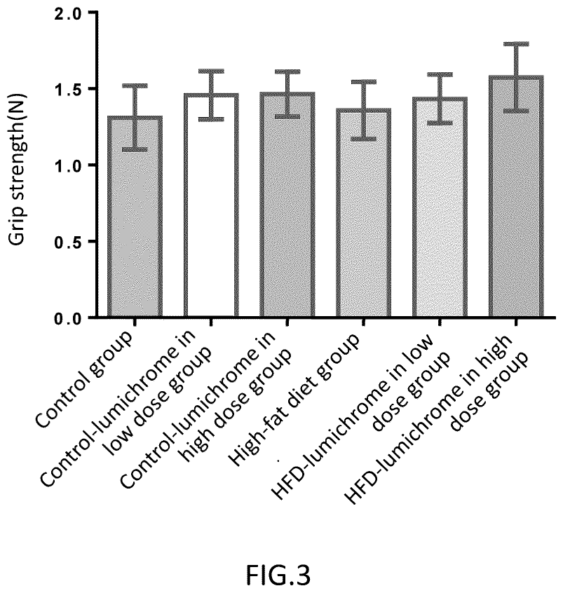

As shown in FIG. 3, all groups, including the Control group, had similar grip strengths, which indicated that (1) providing lumichrome or (2) providing both lumichrome and a high-fat diet did not cause a decrease in muscular endurance of mice. That is, while lumichrome significantly inhibited weight gains of mice resulting from the high-fat diet, such inhibition efficacy was not achieved by reducing the muscle mass of mice.

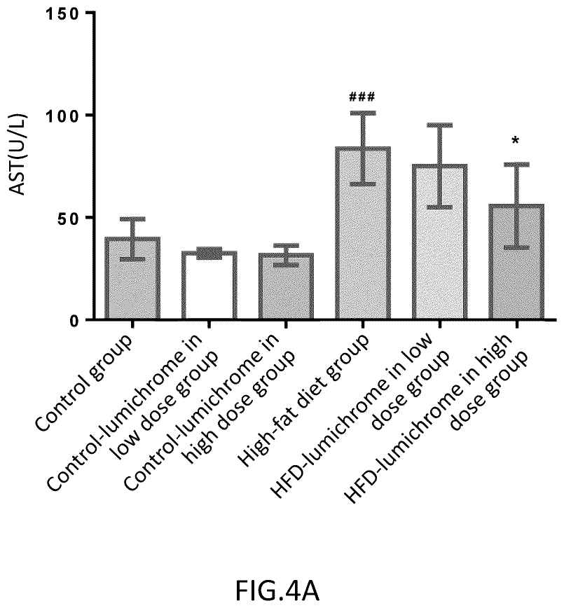

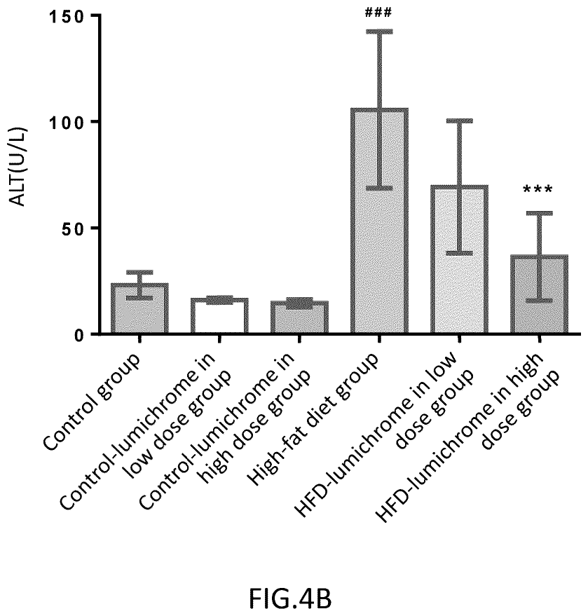

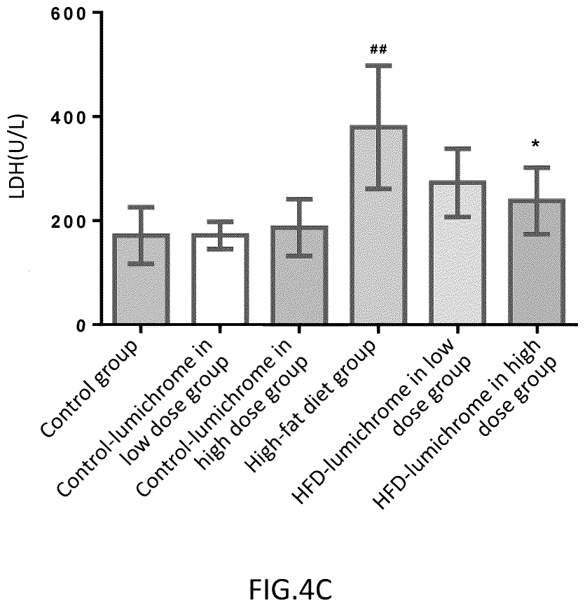

As shown in FIG. 4A to FIG. 4C, the High-fat diet group had the values of AST, ALT, and LDH that were significantly higher than those of the Control group, respectively, so one can find that the high-fat diet increased AST, ALT, and LDH in the serum of mice, which indicated that high-fat diet can cause inflammation and damages in the liver. In contrast, both the HFD-lumichrome in low dose group and the HFD-lumichrome in high dose group had the values of AST, ALT, and LDH that were lower than those of the High-fat diet group, respectively, so one can find that providing lumichrome in both low or high dose can effectively inhibit the increase of AST, ALT and LDH resulting from the high-fat diet. That is, lumichrome can be used to counteract inflammation and damages in the liver resulting from the high-fat diet. Therefore, lumichrome can be used in the treatment or improvement of liver diseases.

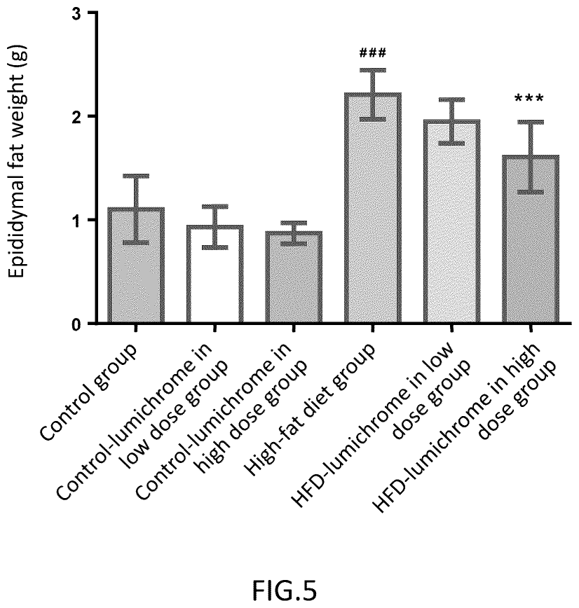

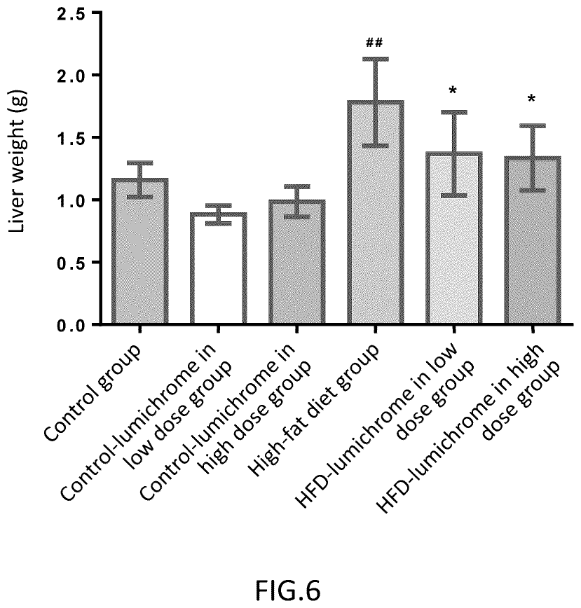

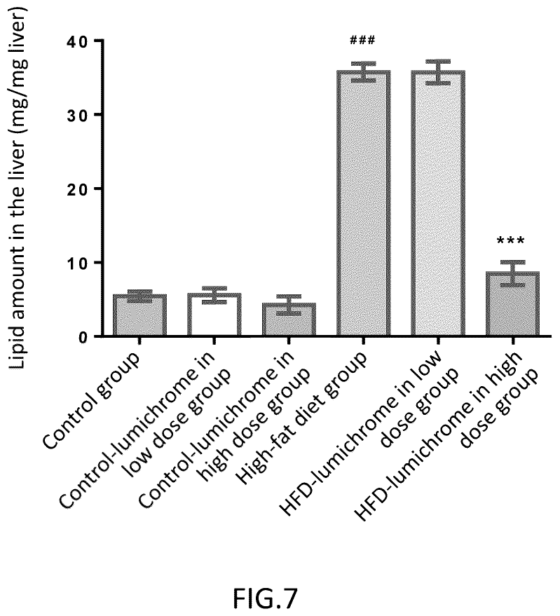

As shown in FIG. 5 to FIG. 7, the High-fat diet group had the values of epididymal fat weight, liver weight and the lipid amount in the liver that were significantly higher than those of the Control group, respectively, so one can find that the high-fat diet increased the visceral fat and body fat of mice.

In contrast, the HFD-lumichrome in low dose group had the values of epididymal fat weight and liver weight that were lower than those of the High-fat diet group, respectively, so one can find that providing lumichrome in low dose can effectively inhibit the increase of epididymal fat weight and liver weight resulting from the high-fat diet.

Further, the HFD-lumichrome in high dose group had the values of epididymal fat weight, liver weight and the lipid amount in the liver that were lower than those of the High-fat diet group, respectively, so one can find that providing lumichrome in high dose can effectively inhibit the increase of epididymal fat weight, liver weight and the lipid amount in the liver resulting from the high-fat diet.

To sum up, one can find that lumichrome can inhibit the increase of both the visceral fat and body fat. Therefore, lumichrome has excellent therapeutic effects on obesity and/or non-alcoholic fatty liver disease related to obesity.

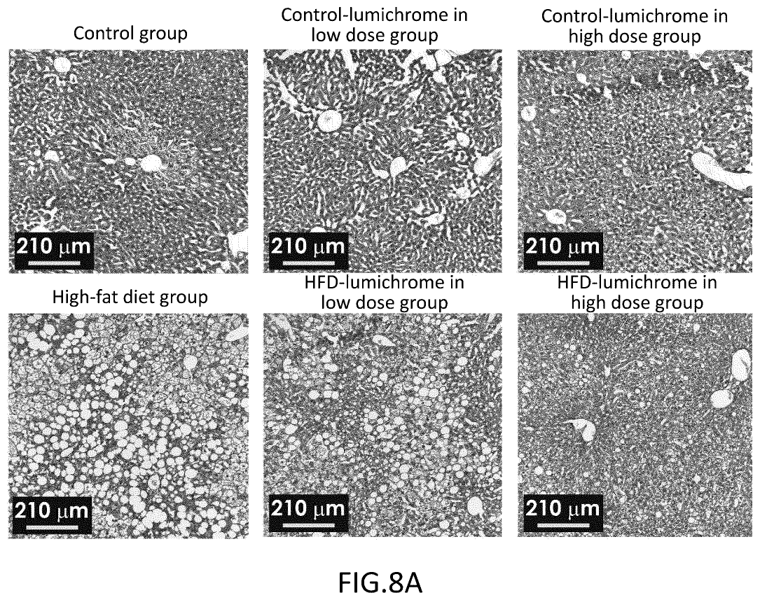

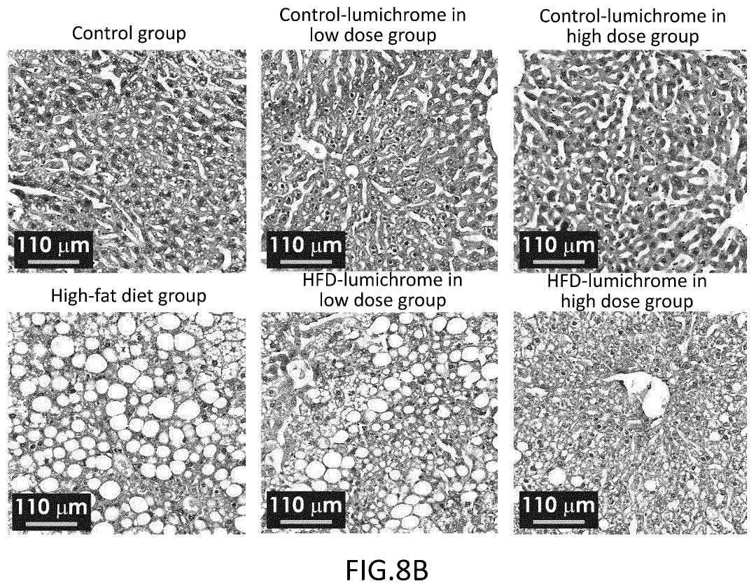

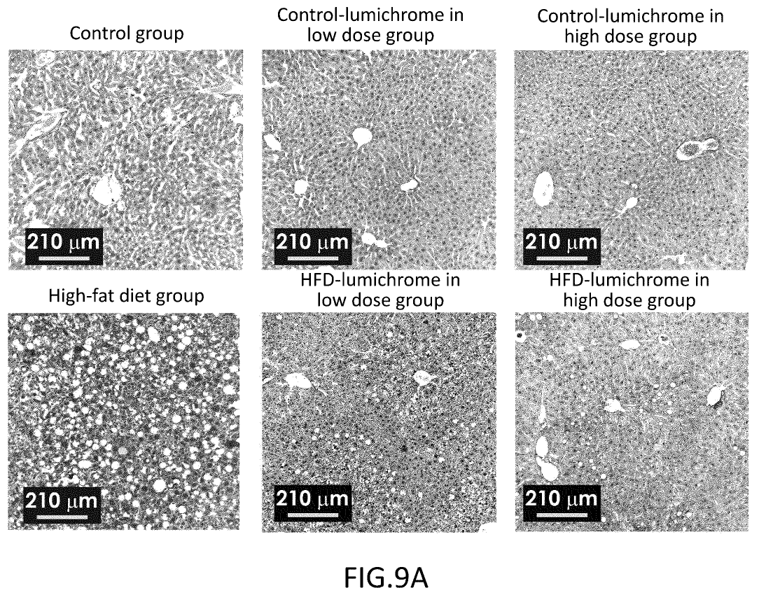

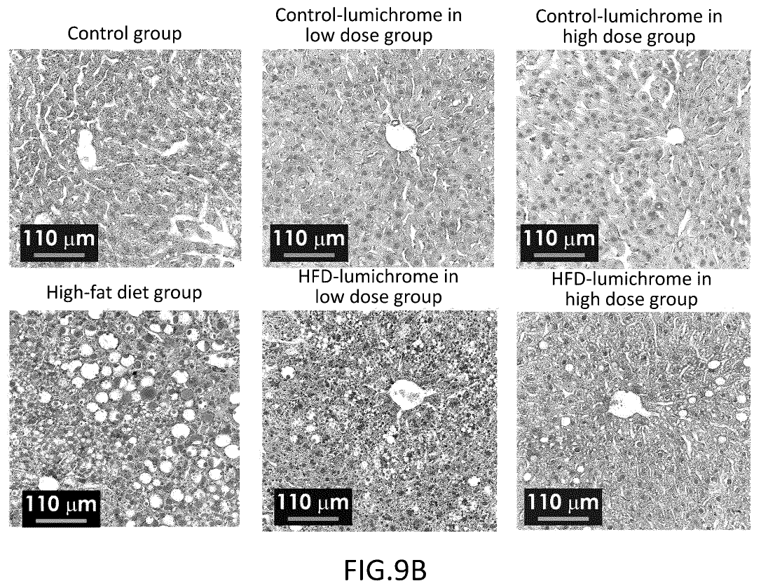

As shown in the liver sections after H/E staining in FIG. 8A and FIG. 8B, and the liver sections after ORO staining in FIG. 9A and FIG. 9B, the High-fat diet group had more lipid droplets accumulation than that of the Control group, so one can find that the high-fat diet induced a large amount of lipid droplets accumulated in the liver of mice.

In contrast, both the HFD-lumichrome in low dose group and the HFD-lumichrome in high dose group had smaller lipid droplets and less lipid droplets accumulation than those of the High-fat diet group, so one can find that providing lumichrome in both low or high dose can (1) shrink the size of lipid droplets in the liver resulting from the high-fat diet, or (2) effectively reduce lipid droplets accumulation in the liver resulting from the high-fat diet. Further, such lipid droplets inhibition efficacy in the HFD-lumichrome in high dose group was significantly better than that in the HFD-lumichrome in low dose group. Therefore, lumichrome has excellent therapeutic effects on obesity and/or non-alcoholic fatty liver disease related to obesity.

For clarification, the large white blanks or cavities in the Control group in FIG. 8A were the sections of blood vessels, and the same existed in all other groups in FIG. 8A, FIG. 8B, FIG. 9A and FIG. 9B.

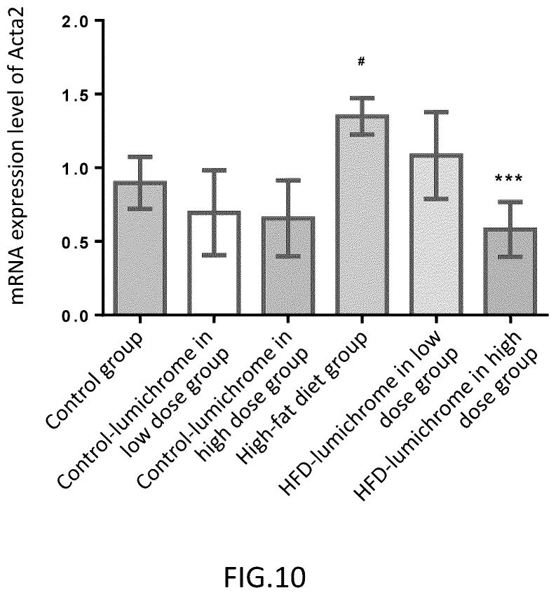

As shown in FIG. 10, the High-fat diet group had a higher mRNA expression of Acta2 than that of the Control group, so one can find that the high-fat diet increased the mRNA expression of Acta2, which relates to liver fibrosis, resulting from the high-fat diet in the liver of mice.

In contrast, both the HFD-lumichrome in low dose group and the HFD-lumichrome in high dose group had a less mRNA expression of Acta2 than that of the High-fat diet group, so one can find that providing lumichrome in both low and high doses can effectively inhibit the mRNA expression increase of Acta2 in the liver of mice resulting from the high-fat diet.

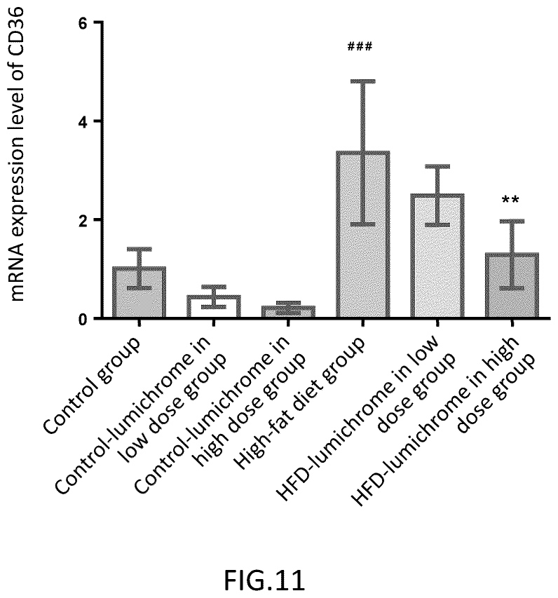

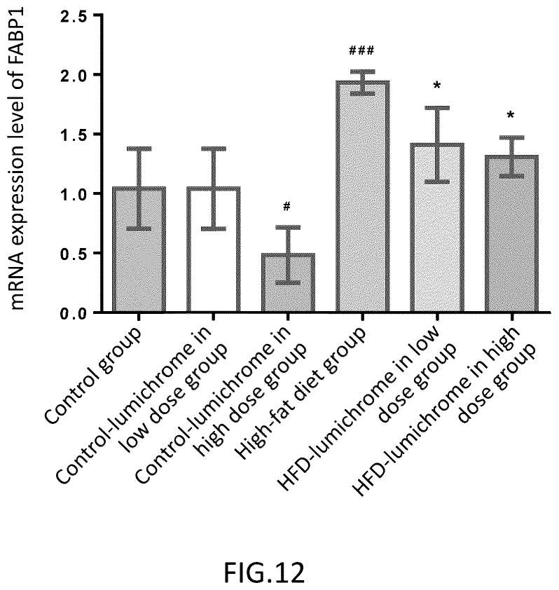

As shown in FIG. 11 and FIG. 12, the High-fat diet group had higher mRNA expressions of both CD36 and FABP1 than those of the Control group, respectively, so one can find that the high-fat diet increased the mRNA expression of both CD36 and FABP1, which both relate to fat uptake by liver cells resulting from the high-fat diet, in the liver of mice.

In contrast, both the HFD-lumichrome in low dose group and the HFD-lumichrome in high dose group had a less mRNA expression of CD36 and FABP1 than that of the High-fat diet group, so one can find that providing lumichrome in both low and high doses can effectively inhibit the increased expression of CD36 and FABP1, which both relate to fat uptake by liver cells, in the liver of mice resulting from the high-fat diet. Therefore, lumichrome has an excellent liver-protective efficacy.

To sum up, without loss of muscle mass or muscular endurance, lumichrome can decrease weight gains, decrease increased body fat, improve fatty liver, and inhibit lipid droplets accumulation in the liver resulting from a high-fat diet. Therefore, lumichrome has excellent therapeutic effects on non-alcoholic fatty liver disease and/or obesity.

The above embodiments are only preferred embodiments of the present invention, not intended to limit the present invention in any aspect. It is apparent to those skilled in the art that various modifications and variations can be made without departing from the scope and spirit of the invention. Although the present invention has been described in terms of specific preferred embodiments, it should be understood that the invention should not be unduly limited to those specific embodiments. In fact, the various modifications that are obvious to those of ordinary skill in the art are also encompassed within the scope of the following claims.

What is claimed is:

1. A method for a treatment or improvement of non-alcoholic fatty liver disease (NAFLD), comprising administering a composite to a subject in need thereof, wherein the composite comprises lumichrome.

2. The method as claimed in claim 1, wherein the composite is a medical composite or a food composite.

3. The method as claimed in claim 2, wherein the medical composite comprises an effective dose of lumichrome and a pharmaceutically acceptable carrier.

4. The method as claimed in claim 1, wherein the subject is a mammal.

5. The method as claimed in claim 4, wherein the mammal is a human.

6. The method as claimed in claim 3, wherein the effective dose of lumichrome is 0.01 mg/kg/day to 2 mg/kg/day.

7. The method as claimed in claim 5, wherein the effective dose of lumichrome is 0.01 mg/kg/day to 2 mg/kg/day.

8. The method as claimed in claim 2, wherein the medical composite is in an enteral dosage form.

9. The method as claimed in claim 8, wherein the enteral dosage form is an oral dosage form.

10. The method as claimed in claim 2, wherein the food composite is a nutraceutical, a dietary supplement or a functional supplement.

11. A method for a treatment or improvement of obesity, comprising administering a composite to a subject in need thereof, wherein the composite comprises lumichrome.

12. The method as claimed in claim 11, wherein the composite is a medical composite or a food composite.

13. The method as claimed in claim 12, wherein the medical composite comprises an effective dose of lumichrome and a pharmaceutically acceptable carrier.

14. The method as claimed in claim 11, wherein the subject is a mammal.

15. The method as claimed in claim 14, wherein the mammal is a human.

16. The method as claimed in claim 13, wherein the effective dose of lumichrome is 0.01 mg/kg/day to 2 mg/kg/day.

17. The method as claimed in claim 15, wherein the effective dose of lumichrome is 0.01 mg/kg/day to 2 mg/kg/day.

18. The method as claimed in claim 12, wherein the medical composite is in an enteral dosage form.

19. The method as claimed in claim 18, wherein the enteral dosage form is an oral dosage form.

20. The method as claimed in claim 12, wherein the food composite is a nutraceutical, a dietary supplement or a functional supplement.


SEQUENCE LISTING (6 sequences)

SEQ ID NO: 1 - DNA, 21 bp, synthetic construct
  source 1..21: mol_type = other DNA
  misc_feature 1..21: note = PCR_primers_fwd_seq of Acta2
gtgaagaggaagacagcacag

SEQ ID NO: 2 - DNA, 21 bp, synthetic construct
  source 1..21: mol_type = other DNA
  misc_feature 1..21: note = PCR_primers_rev_seq of Acta2
gcccattccaaccattactcc

SEQ ID NO: 3 - DNA, 21 bp, synthetic construct
  source 1..21: mol_type = other DNA
  misc_feature 1..21: note = PCR_primers_fwd_seq of CD36
gcgacatgattaatggcacag

SEQ ID NO: 4 - DNA, 22 bp, synthetic construct
  source 1..22: mol_type = other DNA
  misc_feature 1..22: note = PCR_primers_rev_seq of CD36
gatccgaacacagcgtagatag

SEQ ID NO: 5 - DNA, 20 bp, synthetic construct
  source 1..20: mol_type = other DNA
  misc_feature 1..20: note = PCR_primers_fwd_seq of FABP1
tctccggcaagtaccaattg

SEQ ID NO: 6 - DNA, 21 bp, synthetic construct
  source 1..21: mol_type = other DNA
  misc_feature 1..21: note = PCR_primers_rev_seq of FABP1
ttgatgtccttccctttctgg